(19) United States
(12) Patent Application Publication
(10) Pub. No.: US 2004/0121992 A1
(43) Pub. Date: Jun. 24, 2004

(54) Therapeutic methods of reducing cholesterol accumulation

(76) Inventor: Norman B. Javitt, New York, NY (US)

Correspondence Address: KLAUBER & JACKSON, 411 HACKENSACK AVENUE, HACKENSACK, NJ, 07601

(21) Appl. No.: 10/659,764

(22) Filed: Sep. 10, 2003

Related U.S. Application Data
(60) Provisional application No. 60/409,664, filed on Sep. 10, 2002.

(51) Int. Cl. A61K 39/395; A61K 31/56

(52) U.S. Cl. 514/170; 424/130.1; 424/146.1

(57) ABSTRACT

Methods of reducing the cholesterol accumulation in a subject, including methods of reducing cholesterol synthesis and methods of increasing cholesterol degradation. Cholesterol synthesis is inhibited by administering a compound capable of increasing 27-hydroxy-7-dehydrocholesterol and/or 27-hydroxy-8-dehydrocholesterol levels, wherein an increase in 27-hydroxy-7-dehydrocholesterol and/or 27-hydroxy-8-dehydrocholesterol levels results in an inhibition of cholesterol synthesis. Cholesterol degradation is increased by increasing the level of 7α-hydroxylase in extrahepatic tissue and cells.

### GOVERNMENT SUPPORT

The research leading to the present invention was supported in part by research NBLBI Grant No. 63304. The government may have certain rights in the present invention.

### FIELD OF THE INVENTION

This invention relates to methods of modulating the rate of cholesterol synthesis in a mammal. More specifically, the invention relates to treatment of cholesterol-related conditions which are improved or ameliorated by lowering the rate of cholesterol synthesis in a human in need thereof by administration of a 27-hydroxy-7-dehydrocholesterol reductase inhibitor.

### BACKGROUND

Cholesterol is the major steroid constituent of animal tissue, and an essential component of plasma and cell membranes. Cholesterol is a 3-hydroxy sterol having a perhydro-1,2-cyclopenenophenanthrene ring system and an aliphatic side chain at position 17. Because it is insoluble in body fluids, cholesterol must be transported through the bloodstream by carriers such as low-density lipoprotein (LDL).

Excessive accumulation of cholesterol has been implicated as the primary causative factor in a number of diseases, including atherosclerosis, which is characterized by an abnormal hardening and thickening of the arterial walls due to the accumulation and deposition of fatty materials, including cholesterol. This can lead to thrombosis and infarction.

The cholesterol metabolite 26-hydroxycholesterol, now known as 27-hydroxycholesterol, has been previously shown to be associated with cholesterol synthesis (U.S. Pat. No. 4,427,668). More specifically, a reduced level of 27-hydroxycholesterol in the serum was found to be associated with cholesterol build up in the tissues; thus, the administration of 27-hydroxycholesterol was proposed as a method for reducing the rate of cholesterol synthesis in the body. Thereafter, as disclosed in U.S. Pat. No. 4,939,134, it was discovered that 27-amino cholesterol and certain amino-substituted analogs and derivates were more potent inhibitors of cholesterol synthesis and accumulation than 27-hydroxycholesterol. 27-hydroxycholesterol was later found to be effective in reducing the occurrence of restenosis following injury to the blood vessels lumen (U.S. Pat. No. 5,376,652). Individuals with a genetic defect in producing 27-hydroxycholesterol exhibit accelerated atherosclerosis and die early in life of severe coronary artery disease. The molecular basis of this genetic disease is a mutation in the CYP 27 gene, which results in a lack of cholesterol 27-hydroxylase activity.

Kandutsch et al. (1978) Science 201:498, mentioned that oxygenated cholesterol has an inhibitory effect on the proliferation of fibroblasts and lymphocytes in vitro, perhaps by inhibiting hydroxymethylglutaryl coenzyme A reductase (HMG CO-A reductase), the rate-limiting enzyme in cholesterol biosynthesis. The inhibitory effect by oxysterols on vascular smooth muscle cells has been suggested to be a toxic effect (Zhou et al. (1993) Proc. Soc. Exp. Biol. Med. 202:75-80).

Oxysterols, including 24, 25-epoxy cholesterol, 25-hydroxycholesterol, 27-hydroxycholesterol, are now known to also function as ligands for nuclear receptors that modulate other determinants of cholesterol homeostasis (Janowski et al. (1996) Nature 383:728-31). The oxysterol 25R, 26-hydrocholesterol (also referred to as 27-hydroxycholesterol) appears to have a role in preventing cholesterol accumulation in arteries.

Although in humans two different metabolic pathways, beginning with hydroxylation of cholesterol at either the C7α or C27 position of cholesterol (Bjorkhem et al. (2002) J. Biol. Chem. 277:26804-26807) contribute to the production of chenodeoxycholic and cholic acids, it is the former pathway that appears to be specifically activated when bile acids are lost from the enterohepatic circulation. Consonant with the knowledge that cholesterol 7α-hydroxylase activity increases to compensate for a loss of bile acid from the enterohepatic circulation is the knowledge that feeding bile acids to humans markedly lowers cholesterol 7α-hydroxylase activity (Reihner et al. (1989) Gastroenterology 97:1498-1505).

### SUMMARY OF THE INVENTION

The invention provides methods of reducing cholesterol accumulation in a subject in need thereof. In one feature, the invention provides a method of reducing cholesterol synthesis. In another feature, the invention provides a method for increasing cholesterol degradation. Both of these features of the invention are described fully below.

Studies of the regulation of cholesterol synthesis in Smith-Lemli-Opitz syndrome (SLOS) have led to the discovery of a new metabolic route that generates novel endogenous oxysterol ligands. The work described below describes the identification of members of this new class of oxysterols which circulate in plasma and are derived from precursor intermediates in the metabolic pathway for cholesterol synthesis. Smith-Lemli-Opitz syndrome (SLOS) is a genetically determined disease resulting from a mutation in the gene encoding the 7-dehydrocholesterol 7-reductase (DHC7R) enzyme that converts 7-dehydrocholesterol to cholesterol. Neonates suffering from SLOS have very high levels of 7-dehydrocholesterol in plasma, and a decreased cholesterol synthesis.

One feature of the invention is based on the identification of 27-hydroxy metabolite as potent inhibitor of cholesterol synthesis. More specifically, cholesta-5,7-diene-3β-27 diol (also known as 27-hydroxy-7-dehydrocholesterol) is identified as a potent inhibitor of cholesterol synthesis. Further, a second 27-hydroxy metabolite, cholesta-5,8-diene-3β-27 diol (also known as 27-hydroxy-8-dehydrocholesterol) is expected to also act as a potent inhibitor of cholesterol synthesis. One feature of the invention is directed to methods that lower cholesterol accumulation in a subject by providing methods that inhibit cholesterol synthesis. Accordingly, methods are herein provided that increase 27-hydroxy-7-dehydrocholesterol and/or 27-hydroxy-8-dehydro cholesterol to result in inhibition of cholesterol synthesis.

Accordingly, in a first aspect, the invention features a therapeutic method of reducing cholesterol synthesis in a subject in need thereof, comprising administering a therapeutically effective amount of cholesta-5,7-diene-3β-27 diol (27-hydroxy-7-dehydrocholesterol) and/or cholesta-5,8-diene-3β-27 diol (27-hydroxy-8-dehydrocholesterol), wherein cholesterol synthesis is reduced.

In a more specific embodiment, cholest-5,7-diene -3β-27 diol is administered. In another specific embodiment, cholesta-5,8-diene-3β-27 diol is administered. In another specific embodiment, both cholesta-5,7-diene-3β-27 diol and cholesta-5,8-diene-3β-27 diol are administered together.

In a second aspect, the invention features a therapeutic method of reducing cholesterol synthesis in a patient in need thereof, comprising administering a compound capable of inhibiting 27-hydroxy-7-dehydrocholesterol reductase activity, wherein cholesta-5,7-diene-3β-27 diol and/or cholesta-5,8-diene-3β-27 diol levels are increased. In one embodiment, the compound capable of reducing 27-hydroxy-7-dehydrocholesterol reductase activity is an anti-27-hydroxy-7-dehydrocholesterol reductase antibody. In another embodiment, the compound capable of reducing 27-hydroxy-7-dehydrocholesterol reductase activity is a compound which interferes with the synthesis of 27-hydroxy-7-dehydrocholesterol, such as a nucleic acid.

In another embodiment of the method of the invention, a nucleic acid encoding a peptide or protein inhibitor of 27-hydroxy-7-dehydrocholesterol reductase is administered, and in a related embodiment, an antisense sequence or catalytic RNA capable of interfering with the expression of 27-hydroxy-7-dehydrocholesterol reductase is administered. In further embodiments, inhibition of 27-hydroxy-7-dehydrocholesterol reductase activity is achieved with a combination of these approaches.

In a third aspect, the invention features a screening method for identifying agent compounds capable of inhibiting 27-hydroxy-7-dehydrocholesterol reductase activity, comprising measuring the reduction of 27-hydroxy-7-dehydrocholesterol by 27-hydroxy-7-dehydrocholesterol reductase in the presence of a test compound, wherein a compound which reduces 27-hydroxy-7-dehydrocholesterol reductase activity relative to a control is identified as a compound capable of inhibiting 27-hydroxy-7-dehydro-cholesterol reductase activity.

Compounds capable of inhibiting 27-hydroxy-7-dehydrocholesterol reductase activity directly or by interfering with the expression of the enzyme are encompassed in the method of the invention. Accordingly, in a fourth related aspect, the invention features a screening method for identifying agent compounds capable of interfering with the expression of 27-hydroxy-7-dehydrocholesterol reductase activity, comprising determining the level of 27-hydroxy-7-dehydrocholesterol reductase or its encoding mRNA , wherein a compound which reduces 27-hydroxy-7-dehydrocholesterol reductase levels (or its encoding mRNA) relative to a control is identified as a compound capable of inhibiting the expression 27-hydroxy-7-dehydrocholesterol reductase.

In one embodiment, compounds that inhibit the activity or expression of 27-hydroxy-7-dehydrocholesterol reductase are identified in a cell-free assay system. In another embodiment, compounds that inhibit the activity or expression of 27-hydroxy-7-dehydrocholesterol reductase are identified in a cell-based assay system. In another embodiment, agents that inhibit the expression, activity or both the expression and activity of 27-hydroxy-7-dehydrocholesterol reductase are identified in an animal model.

In a fifth aspect, the invention features pharmaceutical compositions useful in the therapeutic method of the invention. More specifically, the invention features a pharmaceutical composition comprising purified cholesta-5,7-diene-3β-27 diol and a pharmaceutically acceptable carrier. In another embodiment, the invention features a pharmaceutical composition comprising purified cholesta-5,8-diene-3β-27 diol, and a pharmaceutically acceptable carrier. In yet another embodiment, the invention features a pharmaceutical composition comprising both cholesta-5,7-diene-3β-27 diol and cholesta-5,8-diene-3β-27 diol, and a pharmaceutically acceptable carrier.

A second feature of the invention is directed to lowering cholesterol accumulation in a subject through activation of a secondary cholesterol breakdown pathway. Most studies of cholesterol 7-α-hydroxylase have found the enzyme is only expressed in the liver. The reaction catalyzed by 7-α-hydroxylase is the initial and rate-limiting step of the primary pathway through which cholesterol is metabolized to bile acids. The 7-α-hydroxylase reaction is autoregulated by end-product inhibition. Thus, during the enterohepatic circulation of bile acids from the intestines back to the liver for secretion into bile, the FXR nuclear receptor is activated, which initiates a chain of events that modulate the promoter region of the CYP 7A1 gene and thus control its transcription.

Because the normal extraction efficiency of the liver for bile acids exceeds 90%, bile acids levels in plasma are much lower than in the portal vein or liver. The present invention provides a novel therapeutic strategy for reducing cholesterol accumulation in a subject by enhancing the metabolism of cholesterol to bile acids in non-hepatic tissue. Non-hepatic cholesterol degradation is obtained by expression of the gene encoding 7-α-hydroxylase in non-hepatic tissue, such as for example, endothelial cells and macrophages. Expression of CYP 7A1 in these nonhepatic tissues would not be autoregulated to the same extent as occurs in the liver because of the very low bile acid levels. Similarly, upregulation of CYP7B1, the oxysterol 7α-hydroxylase normally expressed in macrophages and many other tissues, can enhance bile acid production via the oxysterol metabolic pathway.

Accordingly, in a sixth aspect, the invention provides a method of reducing cholesterol accumulation in a subject by increasing non-hepatic (extrahepatic) cholesterol degradation, comprising increasing the level of 7α-hydroxylase in non-hepatic tissue. In one embodiment, the method comprises providing a nucleic acid construct encoding 7α-hydroxylase to a non-hepatic cell. In one embodiment, the nucleic acid encoding 7α-hydroxylase is the enzyme coding portion of the CYP 7A1 gene. In one embodiment, the non-hepatic cell is an epithelial cell, a macrophage, a skeletal muscle fiber, or a smooth muscle fiber.

In a related seventh aspect, the invention provides a method reducing cholesterol accumulation in a subject by increasing non-hepatic cholesterol degradation cholesterol, comprising increasing the level of oxysterol 7α-hydroxylase in non-hepatic tissue. In one embodiment, the level of oxysterol 7α-hydroxylase in non-hepatic tissue is increased by providing a nucleic acid construct encoding oxysterol 7α-hydroxylase to a non-hepatic cell. In one embodiment, the nucleic acid encoding oxysterol 7α-hydroxylase is the enzyme coding portion of the CYP 7B 1 gene. In one embodiment, the non-hepatic cell is an epithelial cell, a macrophage, a skeletal muscle fiber, or a smooth muscle fiber.

In a related eighth aspect, the invention provides a method of reducing cholesterol accumulation by increasing non-hepatic cholesterol degradation, comprising increasing the levels of 7α-hydroxylase and oxysterol 7α-hydroxylase in non-hepatic tissue, comprising providing a nucleic acid construct encoding 7α-hydroxylase and oxysterol 7α-hydroxylase to a non-hepatic cell. In one embodiment, the nucleic acid encoding oxysterol 7α-hydroxylase is the enzyme coding portion of the CYP 7B1 gene. In one embodiment, the non-hepatic cell is an epithelial cell, a macrophage, a skeletal muscle fiber, or a smooth muscle fiber.

Other objects and advantages will become apparent from a review of the ensuing detailed description taken in conjunction with the following illustrative drawings.

### BRIEF DESCRIPTION OF THE DRAWINGS

FIGS. 1-3 shows the GLC-MS analysis of SLOS plasma. FIG. 1. shows the three peaks detected at 22.0, 22.5 and 23.8 min; FIG. 2 indicates that the first peak has the ions characteristics of 27-hydroxycholesterol; and FIG. 3 indicates the third peak has the ions characteristic of 27-hydroxy-7-dehydro- and 8-dehydrocholesterol.

FIGS. 4-6 is the GLC-MS analysis of cholesta-5,7-diene-3,27-diol (27-hydroxy-7-dehydrocholesterol). FIG. 4 depicts the retention time (23.8 min) of the standard, cholesta-5,7-diene-3β,27-diol prepared by a known method (20). The complete mass spectral pattern of the standard is shown in FIG. 5, and the mass spectral pattern of the peak obtained from a pooled plasma sample from patients with SLOS is shown in FIG. 6.

FIG. 7 shows the correlation of SLOS patient plasma sterol and 27-hydroxysterol levels.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### DEFINITIONS

“Treatment” refers to the administration of medicine or the performance of medical procedures with respect to a patient, for either prophylaxis (prevention) or to cure the infirmity or malady in the instance where the patient is afflicted.

The term “antibody” as used herein includes intact molecules as well as fragments thereof, such as Fa, F(ab′)₂, and Fv, which are capable of binding the epitopic determinant. Antibodies that bind 27-hydroxy-7-dehydrocholesterol reductase can be prepared using intact polypeptides or fragments containing small peptides of interest as the immunizing antigen. The polypeptide or peptide used to immunize an animal can be derived from translated cDNA or chemically synthesized, and can be conjugated to a carrier protein, if desired. Commonly used carriers that are chemically coupled to peptides include bovine serum albumin and thyroglobulin. The coupled peptide is then used to immunize the animal (e.g., a mouse, a rat, or a rabbit).

A “therapeutically effective amount” is an amount of a reagent sufficient to decrease or prevent the symptoms associated with the 27-hydroxy-7-dehydrocholesterol reductase-mediated disorder.

General Description

Studies of the regulation of cholesterol synthesis in Smith-Lemli-Opitz (SLOS) syndrome have led to the identification of a new class of oxysterols that circulate in plasma and are derived from precursor intermediates in the metabolic pathway for cholesterol synthesis. Specifically, the 27-hydroxy metabolites of 7- and 8-dehydrocholesterol (7-DHC and 8-DHC) were identified as potent inhibitors of cholesterol synthesis. The elevated levels of these metabolites in SLOS as compared with normal can be attributed to deficient 3β-hydroxysteroid Δ⁷ -reductase (27-hydroxy-7-dehydrocholesterol reductase) activity.

In addition to their potent effect on cholesterol synthesis, this new class of endogenously occurring oxysterol metabolites can function as ligands for nuclear receptors that modulate cholesterol homeostasis. The nuclear receptors LXRα and LXRβ have been implicated in the control of cholesterol and fatty acid metabolism in multiple cell type. LXRα is expressed primarily in liver, intestine, adipose tissue, and macrophages, whereas LXRβ is expressed in may cell types (Repa et al. (2000) Annu. Rev. Cell Dev. Biol. 16:459-481). In peripheral cells such as macrophages, LXRs seem to coordinate a physiologic response to cellular cholesterol loading. The physiologic ligands for these recptors have been speculated to include specific intermediates in the cholesterol biosynthetic pathway, such as 24(S), 25-epoxycholesterol (Joseph et al. (2002) Proc. Natl. Acad. Sci. USA 99:7604-7609; Janowski et al. (1999) Proc. Natl. Acad. Sci. USA 96:266-271).

Example 1 below describes experiments analyzing plasma samples of children with SLOS. Using gas liquid chromatography-mass spectroscopy (GLC-MS), cholesterol 27-hydroxy metabolites were identified in serum. Using cell culture assays, it was found that 27-hydroxy-7-dehydrocholesterol is a potent inhibitor of cholesterol synthesis.

Therapeutic Uses of the Invention

The invention provides for treatment, amelioration or prevention of cholesterol-related diseases and disorders by administration of a therapeutic agent. Such agents include but are not limited to: agents which promote or prevent expression and/or activity of 27-hydroxy-7-dehydrocholesterol reductase, agents which modulate the activity of 27-hydroxy-7-dehydrocholesterol reductase, agents able to act as antagonists of 27-hydroxy-7-dehydrocholesterol reductase, antibodies able to inhibit the activity of 27-hydroxy-7-dehydrocholesterol reductase, and 27-hydroxy-7-dehydrocholesterol and/or 27-hydroxy-8-dehydrocholesterol.

Therapeutic Compositions

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In one specific embodiment, a non-human mammal is the subject. In another specific embodiment, a human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection by aerosol inhaler.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

The present invention also provides pharmaceutical compositions and formulations. Such compositions comprise a therapeutically effective amount of an agent, e.g., 27-hydroxy-7-dehydrocholesterol and/or 27-hydroxy-8-dehydrocholesterol, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylaamine, 2-ethylamino ethanol, histidine, procaine, etc.

The amount of the compound of the invention which will be effective in the treatment of a disorder that can be ameliorated by inhibition of 27-hydroxy-7-dehydrocholesterol reductase expression can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Administration Methodology

In one embodiment, a nucleic acid comprising a sequence encoding 7α-hydroxylase is administered. In another embodiment, a nucleic acid comprising a sequence encoding oxysterol 7α-hydroxylase is administered. Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspects, the compound comprises a nucleic acid encoding 7α-hydroxylase and/or oxysterol 7α-hydroxylase, and is part of an expression vector that expresses 7α-hydroxylase and/or oxysterol 7α-hydroxylase in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a viral vector that contains a nucleic acid encoding 7α-hydroxylase and/or oxysterol 7α-hydroxylase, for example, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding the enzyme to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are:, Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

Methods for targeting a nucleic acid construct to a specific cell type are known to the art, see for example, White et al. (2001) Neuron 31:699-711, and White et al. (2001) Current Biology 11:R1041-R1053, both of which publications are herein specifically incorporated by reference in their entirety.

Therapeutic uses of inhibitors of 27-hydroxy-7-dehydrocholesterol reductase.

The invention provides for treatment of diseases and disorders which can be ameliorated by a reduction of cholesterol synthesis, by administration of a therapeutic compound capable of inhibiting 27-hydroxy-7-dehydrocholesterol reductase activity or expression, and/or a compound able to increase the levels of 27-hydroxy-7-dehydrocholesterol and/or 27-hydroxy-8-dehydrocholesterol.

Compounds useful for this purpose include but are not limited to anti-27-hydroxy-7-dehydrocholesterol reductase antibodies (and fragments and derivatives containing the binding region thereof), 27-hydroxy-7-dehydrocholesterol reductase antisense or ribozyme nucleic acids, and nucleic acids encoding dysfunctional 27-hydroxy-7-dehydrocholesterol reductases that are used to “knockout” endogenous 27-hydroxy-7-dehydrocholesterol reductase function by homologous recombination (see, e.g., Capecchi, 1989, Science 244:1288-1292). Other compounds that inhibit 27-hydroxy-7-dehydrocholesterol reductase function can be identified by use of known in vitro assays, e.g., assays for the ability of a test compound to inhibit binding of a 27-hydroxy-7-dehydrocholesterol reductase to another protein or a binding partner, or to inhibit a known 27-hydroxy-7-dehydrocholesterol reductase function. Preferably such inhibition is assayed in vitro or in cell culture, but genetic assays may also be employed. Preferably, suitable in vitro or in vivo assays are utilized to determine the effect of a specific compound and whether its administration is indicated for treatment of the affected tissue, as described in more detail below.

In a specific embodiment, a compound that inhibits 27-hydroxy-7-dehydrocholesterol reductase function is administered therapeutically or prophylactically to a subject in whom a decreased level of cholesterol biosynthesis is desired. Methods standard in the art can be employed to measure the decrease in 27-hydroxy-7-dehydrocholesterol reductase level or function, and/or decrease in cholesterol biosynthesis. Preferred 27-hydroxy-7-dehydrocholesterol reductase inhibitor compositions include small molecules, i.e., molecules of 1000 daltons or less. Such small molecules can be identified by the screening methods described herein.

27-Hydroxy-7-dehydrocholesterol reductase antibodies.

In one embodiment wherein inhibition of the 27-hydroxy-7-dehydrocholesterol reductase is desirable, one or more antibodies each specifically binding to the 27-hydroxy-7-dehydrocholesterol reductase thereof are administered alone or in combination with one or more additional therapeutic compounds or treatments.

Preferably, a biological product such as an antibody is allogeneic to the subject to which it is administered. In a preferred embodiment, a human 27-hydroxy-7-dehydrocholesterol reductase or functionally active fragment thereof is administered to a human subject for therapy (e.g. to ameliorate symptoms or to retard onset or progression) or prophylaxis.

27-Hydroxy-7-dehydrocholesterol reductase antisense nucleic acids.

In a specific embodiment, 27-hydroxy-7-dehydrocholesterol reductase expression is inhibited by use of 27-hydroxy-7-dehydrocholesterol reductase antisense nucleic acids. The present invention provides the therapeutic or prophylactic use of nucleic acids comprising at least six nucleotides that are antisense to a gene or cDNA encoding a 27-hydroxy-7-dehydrocholesterol reductase or a portion thereof. As used herein, a 27-hydroxy-7-dehydrocholesterol reductase “antisense” nucleic acid refers to a nucleic acid capable of hybridizing by virtue of some sequence complementarity to a portion of an RNA (preferably mRNA) encoding a 27-hydroxy-7-dehydrocholesterol reductase. The antisense nucleic acid may be complementary to a coding and/or noncoding region of an mRNA encoding a 27-hydroxy-7-dehydrocholesterol reductase.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the 27-hydroxy-7-dehydrocholesterol reductase antisense nucleic acids of the invention in a pharmaceutically acceptable carrier, as described infra.

In another embodiment, the invention provides methods for inhibiting the expression of a 27-hydroxy-7-dehydrocholesterol reductase nucleic acid sequence in a prokaryotic or eukaryotic cell comprising providing the cell with an effective amount of a composition comprising an 27-hydroxy-7-dehydrocholesterol reductase antisense nucleic acid of the invention.

The 27-hydroxy-7-dehydrocholesterol reductase antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al. (1989) Proc. Natl. Acad. Sci. USA 86:6553-6556; Lemaitre et al. (1987) Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al. (1988) BioTechniques 6:958-976) or intercalating agents (see, e.g., Zon (1988) Pharm. Res. 5:539-549).

In a preferred aspect of the invention, a 27-hydroxy-7-dehydrocholesterol reductase antisense oligonucleotide is provided, preferably of single-stranded DNA. The oligonucleotide may be modified at any position on its structure with substituents generally known in the art.

The 27-hydroxy-7-dehydrocholesterol reductase antisense oligonucleotide may comprise at least one of the following modified base moieties: 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta- D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the 27-hydroxy-7-dehydrocholesterol reductase antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the 27-hydroxy-7-dehydrocholesterol reductase antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the 27-hydroxy-7-dehydrocholesterol reductase antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a 27-hydroxy-7-dehydrocholesterol reductase, preferably a human gene encoding a 27-hydroxy-7-dehydrocholesterol reductase. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.) with the RNA, forming a stable duplex; in the case of double-stranded 27-hydroxy-7-dehydrocholesterol reductase antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding a 27-hydroxy-7-dehydrocholesterol reductase it may contain and still form a stable duplex (or triplex, as the case may be). One skilled in the art can ascertain a tolerable degree of mismatch by use of standard procedures to determine the melting point of the hybridized complex.

The 27-hydroxy-7-dehydrocholesterol reductase antisense nucleic acids can be used to inhibit 27-hydroxy-7-dehydrocholesterol reductase expression in a patient who would benefit from a decreased cholesterol synthesis. In a preferred embodiment, a single-stranded DNA antisense 27-hydroxy-7-dehydrocholesterol reductase oligonucleotide is used. Cell types which express or overexpress RNA encoding a 27-hydroxy-7-dehydrocholesterol reductase can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with a 27-hydroxy-7-dehydrocholesterol reductase-specific nucleic acid (e.g., by Northern hybridization, dot blot hybridization, in situ hybridization), observing the ability of RNA from the cell type to be translated in vitro into a 27-hydroxy-7-dehydrocholesterol reductase, immunoassay, etc. In a preferred aspect, primary tissue from a subject can be assayed for 27-hydroxy-7-dehydrocholesterol reductase expression prior to treatment, e.g., by immunocytochemistry or in situ hybridization.

Pharmaceutical compositions of the invention, comprising an effective amount of a 27-hydroxy-7-dehydrocholesterol reductase antisense nucleic acid in a pharmaceutically acceptable carrier, can be administered to a subject in need thereof.

The amount of 27-hydroxy-7-dehydrocholesterol reductase antisense nucleic acid which will be effective in inhibiting 27-hydroxy-7-dehydrocholesterol reductase expression can be determined by standard clinical techniques.

In a specific embodiment, pharmaceutical compositions comprising one or more 27-hydroxy-7-dehydrocholesterol reductase antisense nucleic acids are administered via liposomes, microparticles, or microcapsules. In various embodiments of the invention, such compositions may be used to achieve sustained release of the 27-hydroxy-7-dehydrocholesterol reductase antisense nucleic acids.

Inhibitory ribozyme and triple helix approaches.

In another embodiment, cholesterol-related disorders may be ameliorated by decreasing the level of a 27-hydroxy-7-dehydrocholesterol reductase or 27-hydroxy-7-dehydrocholesterol reductase activity by using gene sequences encoding the 27-hydroxy-7-dehydrocholesterol reductase in conjunction with well-known gene “knock-out,” ribozyme or triple helix methods to decrease gene expression of a 27-hydroxy-7-dehydrocholesterol reductase. In this approach ribozyme or triple helix molecules are used to modulate the activity, expression or synthesis of the gene encoding the 27-hydroxy-7-dehydrocholesterol reductase, and thus to ameliorate the symptoms of cholesterol-related disease. Such molecules may be designed to reduce or inhibit expression of a mutant or non-mutant target gene. Techniques for the production and use of such molecules are well known to those of skill in the art.

Screening Assays

The invention provides methods for identifying agents (e.g., candidate compounds or test compounds) that are capable of inhibiting the expression or activity of 27-hydroxy-7-dehydrocholesterol reductase. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. NatI. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

In one embodiment, agents that inhibit 27-hydroxy-7-dehydrocholesterol reductase are identified in a cell-free assay system. In accordance with this embodiment, a native or recombinant 27-hydroxy-7-dehydrocholesterol reductase is contacted with a candidate compound or a control compound and the ability of the candidate compound to inhibit the enzyme is determined. If desired, this assay may be used to screen a plurality (e.g. a library) of candidate compounds. In more specific embodiments, the 27-hydroxy-7-dehydrocholesterol reductase enzyme is is first immobilized, by, for example, contacting the enzyme with an immobilized antibody which specifically recognizes and binds it, or by contacting a purified preparation of the enzyme with a surface designed to bind proteins. The 27-hydroxy-7-dehydrocholesterol reductase may be partially or completely purified (e.g., partially or completely free of other polypeptides) or part of a cell lysate. Further, the enzyme may be a fusion protein comprising the enzyme and a domain such as glutathionine-S-transferase. Alternatively, the enzyme may be biotinylated using techniques well known to those of skill in the art (e.g., biotinylation kit, Pierce Chemicals; Rockford, Ill.). The ability of the candidate compound to inhibit 27-hydroxy-7-dehydrocholesterol reductase can be can be determined by methods known to those of skill in the art.

In another embodiment, agents that inhibit 27-hydroxy-7-dehydrocholesterol reductase are identified in a cell-based assay system. In accordance with this embodiment, cells expressing oxysterol 7-redutase activity are contacted with a candidate compound or a control compound and the ability of the candidate compound to inhibit the enzyme acitivity is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express 27-hydroxy-7-dehydrocholesterol reductase endogenously or be genetically engineered to express the enzyme. In certain instances, the enzyme or the candidate compound is labeled, for example with a radioactive label (such as ³²p, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between the enzyme and a candidate compound. The ability of the candidate compound to inhibit 27-hydroxy-7-dehydrocholesterol reductase can be determined by methods known to those of skill in the art.

In another embodiment, agents capable of modulating, e.g., down-regulating the expression of 27-hydroxy-7-dehydrocholesterol reductase are identified by contacting cells (e.g., cells of prokaryotic origin or eukaryotic origin) expressing the enzyme with a candidate compound or a control compound (e.g., phosphate buffered saline (PBS)) and determining the expression of the enzyme or mRNA encoding the enzyme. The level of expression of 27-hydroxy-7-dehydrocholesterol reductase or mRNA encoding the enzyme in the presence of the candidate compound is compared to the level of expression in the absence of the candidate compound (e.g., in the presence of a control compound). The candidate compound can then be identified as a modulator of the expression of 27-hydroxy-7-dehydrocholesterol reductase based on this comparison. For example, when expression of 27-hydroxy-7-dehydrocholesterol reductase or its mRNA is significantly less in the presence of the candidate compound than in its absence, the candidate compound is identified as an inhibitor of the expression of the enzyme or its mRNA. The level of expression of the enzyme or the mRNA that encodes it can be determined by methods known to those of skill in the art. For example, mRNA expression can be assessed by Northern blot analysis or RT-PCR, and protein levels can be assessed by western blot analysis.

In another embodiment, agents that inhibit 27-hydroxy-7-dehydrocholesterol reductase activity are identified by contacting a preparation containing the enzyme, or cells (e.g., prokaryotic or eukaryotic cells) expressing the enzyme with a test compound or a control compound and determining the ability of the test compound to inhibit the activity of 27-hydroxy-7-dehydrocholesterol reductase. The activity of the enzyme can be assessed by detecting induction of a cellular signal transduction pathway of the 27-hydroxy-7-dehydrocholesterol reductase (e.g., downstream metabolites) (e.g., 27-hydroxycholesterol), detecting catalytic or enzymatic activity of the target on a suitable substrate, detecting the induction of a reporter gene, or detecting a cellular response. Based on the present description, techniques known to those of skill in the art can be used for measuring these activities (see, e.g., U.S. Pat. No. 5,401,639, which is incorporated herein by reference). The candidate compound can then be identified as a inhibitor of the activity of 27-hydroxy-7-dehydrocholesterol reductase by comparing the effects of the candidate compound to the control compound. Suitable control compounds include phosphate buffered saline (PBS) and normal saline (NS).

In another embodiment, agents that inhibit the expression, activity or both the expression and activity of 27-hydroxy-7-dehydrocholesterol reductase are identified in an animal model. Examples of suitable animals include, but are not limited to, mice, rats, rabbits, monkeys, guinea pigs, dogs and cats. In specific embodiments, the animal used represent a model of human cholesterol synthesis. In accordance with this embodiment, the test compound or a control compound is administered (e.g., orally, rectally or parenterally such as intraperitoneally or intravenously) to a suitable animal and the effect on the expression, activity or both expression and activity of 27-hydroxy-7-dehydrocholesterol reductase is determined. Changes in the expression of 27-hydroxy-7-dehydrocholesterol reductase can be assessed by the methods outlined above.

### EXAMPLES

The following examples are put forth so as to provide those of ordinary skill in the art with a complete disclosure and description of how to make and use the therapeutic methods of the invention and compounds and pharmaceutical compositions, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Determination of oxysterol metabolites in SLOS patients.

Plasma samples from SLOS patients were analyzed using the method of Reiss et al. (1994) J. Lipid Res. 35, 1026-30) with the following modifications. 7-DHC, the precursor of Vitamin D, is known to undergo structural modification on exposure to ultraviolet light. Accordingly, all routine procedures were done in rooms where the laboratory benches were not in direct sunlight and the artificial lighting was off. Chromatographic tanks were kept in closed cabinets. Choestane-triols were separated from cholestane-diols by TLC prior to GLC-MS anaysis using a solvent system of chloroform:acetone (4:1). Trimethylsilyl rather than acetate derivatives were prepared. Preparation of cholesta-5,7-diene-3β, 27-diol by the method of Kim et al. (1989) J. Lipid Res. 30, 247-61 provided an authentic standard to compare with the detected peak in serum. Based on the knowledge that both 8-DHC and 7-DHC elute either with or shortly after cholesterol, it was expected that the 27-hydroxy metabolites would show a similar relationship.

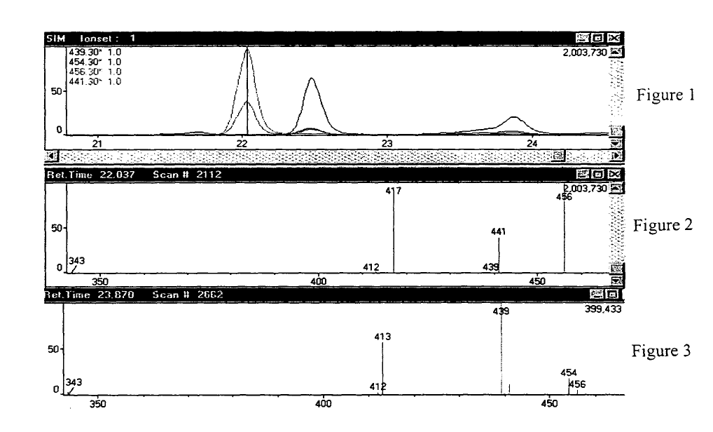

The results are shown in FIGS. 1-3. The trimethylsilyl ether derivatives were prepared from an eluate (Rf=0.52) of the sterol fraction of plasma after thin-layer chromatography on silica gel. Because TLC does not separate 27-hyroxycholesterol from 27-hydroxy-7-dehydrocholesterol and 27-hydroxy-8-dehydrocholesterol they are obtained as a single eluate free of cholesterol (Rf=0.79) and cholest-5-ene-3β, 7α, 27-triol (Rf=0.22). To detect 27-hydroxycholesterol the ion m/z=456 (molecular ion=m/z 546-90) and the (−) 15 ion, m/z=441 were programmed. For 27-hydroxy-7- and 8-dehydrocholesterol the corresponding mn/z=454 and (−) 15 ion, m/z=439 were programmed. As depicted in FIG. 1, three peaks were detected at 22.0, 22.5 and 23.8 min. FIG. 2 indicates that the first peak has the ions characteristics of 27-hydroxycholesterol. FIG. 3 indicates the third peak has the ions characteristic of 27-hydroxy-7-dehydro- and 8-dehydrocholesterol.

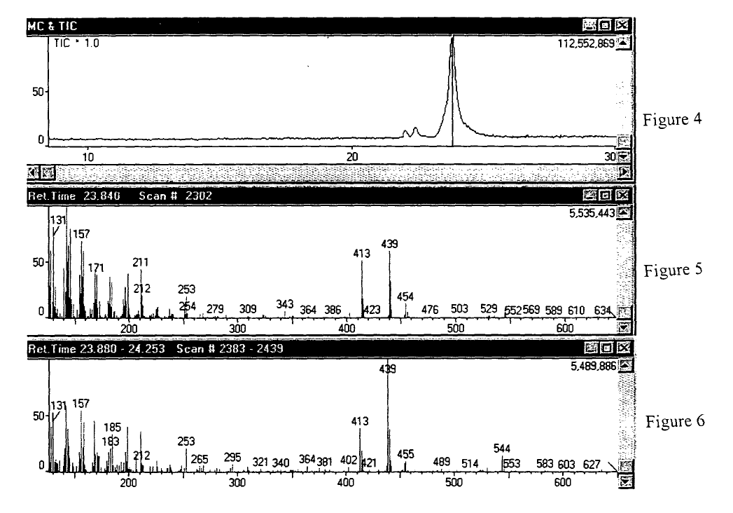

FIG. 4 depicts the retention time (23.8 min) of the standard, cholesta-5,7-diene-3β, 27-diol prepared by the method of Kim et al. (1989) J. Lipid Res. 30, 247-61. The complete mass spectral pattern of the standard is shown in FIG. 5, and the mass spectral pattern of the peak obtained from a pooled plasma sample from patients with SLOS is shown in FIG. 6.

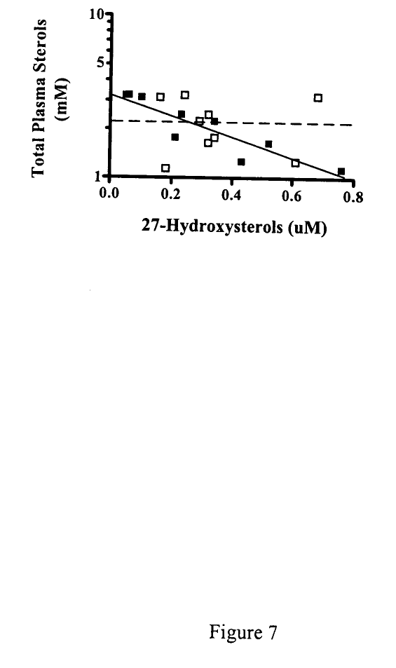

FIG. 7 shows the correlation of SLOS patient plasma sterol and 27-hydroxysterol levels. Total plasma sterol levels represents the sum of cholesterol, 7-DHC and 8-DHC determined by GC/MS. Open squares represent 27-hydroxysterol levels and closed squares represent the level of 27-hydroxydehydrocholesterol levels in corresponding serum samples. Linear regression analysis of the logarithm of total plasma sterols demonstrated a high degree of correlation (r 2=0.81) with 27-dehydrocholesterol levels (solid line); whereas, linear regression analysis showed a poor correlation (r 2 0.001) between the logarithm of total plasma sterols and 27-hydroxycholesterol levels (dashed line).

As shown in Table 1, the amount of 27-hydroxycholesterol was quantified and the amounts of 27-hydroxy-7-dehydro- and 8-dehydrocholesterol in the serum of normal individuals and patients with SLOS syndrome using deuterated 27-hydroxycholesterol as an internal standard (Javitt et al. (1981) J. Biol. Chem. 256:12644-6). The concentration of the 27-hydroxy metabolites of both 7-DHC and 8-DHC were found to be markedly greater than controls. Although we could detect 27-hydroxy-7-dehydrocholesterol in 1.0 ml aliquots of normal serum, we did not detect 27-hydroxy-8-dehydrocholesterol.

TABLE 1
Concentration of 27-hydroxy metabolites*
In SLOS and control plasma

| | 27-hydroxy | 27-hydroxy | 27-hydroxy |
|---|---|---|---|
| | Cholesterol | cholesta-5,8-dienediol | cholesta-5,7-dienediol |
| Patient | uM | uM | uM |
| 1 | 0.18 | 0.51 | 0.25 |
| 2 | 0.16 | 0.07 | 0.03 |
| 3 | 0.34 | 0.14 | 0.07 |
| 4 | 0.32 | 0.37 | 0.15 |
| 5 | 0.32 | 0.14 | 0.09 |
| 6 | 0.24 | 0.04 | 0.02 |
| 7 | 0.28 | 0.11 | 0.03 |
| 8 | 0.68 | 0.04 | 0.01 |
| 9 | 0.29 | 0.26 | 0.08 |
| 10 | 0.61 | 0.30 | 0.13 |
| Control | uM | | pM |
| 1 | 0.41 | none | 2.0 |
| 2 | 0.26 | none | 2.4 |
| 3 | 0.38 | none | 1.0 |

*Systematic names are cholest-5-ene-3 β, 27-diol (27-hydroxycholesterol), cholesta-5,8-diene-3 β, 27-diol (27-hydroxy-5,8-dienediol), cholesta-5,7-diene-3 β, 27-diol

The effect of 27-hydroxy-7-dehydrocholesterol on cholesterol synthesis was studied in CHO cells (Table 2). SLOS fibroblasts were plated at a density of 375,000 cells in a T-25 flask in DMEM containing 10% fetal bovine serum. After 24 hours, the media was changed to McCoy's 5A containing 7.5% lipoprotein deficient serum and 25% D2O. To these cultures, either 5 μM of 27-hydroxycholesterol or 27-hydroxy-7-dehydrocholesterol in hydroxypropyl-β-cyclodextrin were added. Control cultures had only the vehicle added. Media was changed on day 3 and cell pellets were obtained on day 5. Newly synthesized 7-DHC was determined by a combination of GLC-FID and GLC-MS. At a medium concentration of 5 μM 27-hydroxy-7-dehydrocholesterol, the synthesis rate of 7-DHC fell to less than 2 ng/day/T-25 flask in each cell line. Much of the added intermediate underwent reduction at C7 to 27-hydroxycholesterol. Nevertheless, inhibition of cholesterol synthesis was slightly greater than when an equimolar amount of 27-hydroxycholesterol was added to cell culture. To prove the biological activity of 27-hydroxy-7-dehydrocholesterol, we determined 7-DHC synthesis in fibroblasts from two SLOS patients with null mutations (Genotypes for A2SLO and 4350SLO fibroblasts are IVS8-1G>C/IVS8-1G>C and W151X/IVS8-1G>C respectively), A2SLO and 4350SLO, which did not metabolize 27-hydroxy-7-dehydrocholesterol to 27-hydroxycholesterol (data not shown). In untreated cultures, the rates of 7-DHC synthesis were 326±8 and 1414±88 ng/day/T-25 flask (mean±SD) for A2SLO and 4350SLO respectively.

TABLE 2
Effect of cholesta-5,7-diene-27ol on cholesterol synthesis in CHO cells.

| | | | | | | | | ug | cell |
|---|---|---|---|---|---|---|---|---|---|
| | COPROSTANOL | CHOLESTEROL | | ug | | | % new | new | protein |
| Sample | m/z 370 | m/z 368 | 368/370 | cholest. | m/z 372 | 372/368 | chol | chl | mg |
| control 1 | 3595597 | 19967882 | 5.55 | 27.8 | 1844818 | 0.092 | 24.0 | 6.6 | 0.89 |
| control 2 | 4791196 | 28448061 | 5.94 | 29.7 | 2048530 | 0.197 | 19.7 | 5.9 | 0.90 |
| 5 uM | 1619046 | 10746158 | 6.63 | 33.1 | 118666 | 0.011 | 3.6 | 1.2 | 0.90 |
| 27OH | | | | | | | | | |
| 5 uM | 3448190 | 20628855 | 5.98 | 29.9 | 277379 | 0.013 | 4.4 | 1.3 | 0.92 |
| 27OH | | | | | | | | | |
| 5 uM | 5509271 | 32516308 | 5.902 | 29.5 | 258841 | 0.0079 | 2.6 | 0.78 | 1.0 |
| diene | | | | | | | | | |
| 5 uM | 1115415 | 7265024 | 6.51 | 32.6 | 63723 | 0.0087 | 2.9 | 0.95 | 1.0 |
| diene | | | | | | | | | |

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

We claim:

1. A method of reducing cholesterol synthesis in a patient in need thereof, comprising administering a compound capable of increasing cholesta-5,7-diene-3β-27 diol (27-hydroxy-7-dehydrocholesterol) levels, thereby resulting in a reduction of cholesterol synthesis.

2. The method of claim 1, wherein the compound administered is 27-hydroxy-7-dehydrocholesterol.

3. The method of claim 1, wherein the compound administered increases both 27-hydroxy-7-dehydrocholesterol and cholesta-5,8-diene-3μ-27 diol (27-hydroxy-8-dehydrocholesterol).

4. The method of claim 3, wherein the compound administered is 27-hydroxy-8-dehydrocholesterol.

5. The method of claim 3, wherein the compound administered is a combination of 27-hydroxy-7-dehydrocholesterol and 27-hydroxy-8-dehydrocholesterol.

6. A method of reducing cholesterol synthesis in a patient in need thereof, comprising administering a compound capable of reducing 27-hydroxy-7-dehydrocholesterol reductase activity, resulting in an inhibition of cholesterol synthesis.

7. The method of claim 6, wherein the compound capable of reducing 27-hydroxy-7-dehydrocholesterol reductase is an inhibitor of 27-hydroxy-7-dehydrocholesterol reductase.

8. The method of claim 7, wherein the inhibitor of 27-hydroxy-7-dehydrocholesterol reductase is an anti-27-hydroxy-7-dehydrocholesterol reductase antibody.

9. The method of claim 6, wherein the compound capable of reducing 27-hydroxy-7-dehydrocholesterol reductase is a compound which interferes with the synthesis of 27-hydroxy-7-dehydrocholesterol reductase.

10. The method of claim 6, wherein the compound which interferes with the synthesis of 27-hydroxy-7-dehydrocholesterol reductase is a nucleic acid.

11. The method of claim 10, wherein the nucleic acid encoding a peptide or protein inhibitor of 27-hydroxy-7-dehydrocholesterol reductase.

12. The method of claim 10, wherein the nucleic acid is an antisense sequence or catalytic RNA capable of interfering with the expression of 27-hydroxy-7-dehydrocholesterol reductase.

13. A method of reducing cholesterol synthesis in a patient in need thereof, comprising administering a compound capable of increasing 27-hydroxy-7-dehydrocholesterol and 27-hydroxy-8-dehydrocholesterol levels, thereby resulting in a reduction of cholesterol synthesis.

14. The method of claim 13, wherein the compound capable of increasing 27-hydroxy-7-dehydrocholesterol and 27-hydroxy-8-dehydrocholesterol levels is a compound capable of reducing 27-hydroxy-7-dehydrocholesterol reductase activity.

15. The method of claim 13, wherein the compound is cholesta-5,7-diene-3μ-27 diol (27-hydroxy-7-dehydrocholesterol) or cholesta-5,8-diene-3μ-27 diol (27-hydroxy-8-dehydrocholesterol).

16. A screening method for identifying agent compounds capable of inhibiting 27-hydroxy-7-dehydrocholesterol reductase activity, comprising measuring enzymatic activity 27-hydroxy-7-dehydrocholesterol reductase in the presence of a test compound, wherein a compound which reduces 27-hydroxy-7-dehydrocholesterol reductase activity relative to a control is identified as a compound capable of inhibiting 27-hydroxy-7-dehydrocholesterol reductase activity.

17. A screening method for identifying agent compounds capable of interfering with the expression of 27-hydroxy-7-dehydrocholesterol reductase activity, comprising determining the level of 27-hydroxy-7-dehydrocholesterol reductase or its encoding mRNA , wherein a compound which reduces 27-hydroxy-7-dehydrocholesterol reductase levels (or its encoding mRNA) relative to a control is identified as a compound capable of inhibiting the expression 27-hydroxy-7-dehydrocholesterol reductase.

18. A method of increasing cholesterol degradation, comprising:
(a) providing a nucleic acid construct encoding 7α-hydroxylase to an extrahepatic cell; and
(b) expressing the nucleic acid of step (a) in the extrahepatic cell, wherein the level of 7α-hydroxylase is increased.

19. The method of claim 18, wherein the extrahepatic cell is selected from the group consisting of an epithelial cell, a skeletal muscle fiber, and a smooth muscle fiber.

20. The method of claim 18, wherein the nucleic acid construct further encodes oxysterol 7α-hydroxylase.
